(19) United States
(12) Patent Application Publication
(10) Pub. No.: US 2005/0281859 A1
(43) Pub. Date: Dec. 22, 2005

(54) Method and apparatus for forming objects

(76) Inventors: David Philip Knight, Winchester (GB); Larissa Pinnock, Newbury (GB)

Correspondence Address: Rashida A. Karmali, Ph.D., 99 Wall Street, 13th Floor, New York, NY, 10005, US

(21) Appl. No.: 11/158,842

(22) Filed: Jun. 22, 2005

(30) Foreign Application Priority Data
Dec. 23, 2002 (GB) GB 0229993.1

(51) Int. Cl. A61F 2/00

(52) U.S. Cl. 424/423; 264/109

(57) ABSTRACT

The invention utilizes the sol-gel transition behavior of certain proteins for forming objects. The invention is directed to a method and apparatus for forming an object from a feedstock made of a protein solution, the protein undergoing a sol-gel transition and comprising the following steps: a first step of adjusting the conditions to cause the feedstock to flow to form the object from with the protein substantially in the sol state, a second step of adjusting the conditions of the feedstock either to gel the feedstock or bring it close to the sol-gel transition point. Particularly preferred proteins are spidroin and fibroin, while one of the conditions of the feedstock will be adjusting the pH of the solution e.g., with a carboxylic acid.

### TECHNICAL FIELD

The present invention is directed to a method for forming objects from a polymer that undergoes a sol-gel transition. The invention is also directed to an apparatus for forming such objects.

### BACKGROUND

Methods of producing filaments, fibres or moulded objects have been known in the art for a long time. For example, spinning techniques are used to produce fibres from polymer solutions. British patent specification GB-A-441 440 (Ziegrer) discloses one technique in which filaments are produced by passing a liquid raw, material through a porous porcelain tube. The filaments emerge from the end of the porous porcelain tube in this disclosure. An operative medium is introduced into the porous porcelain tube through the pores of the tube.

There is currently considerable interest in the development of improved processes and apparatus to enable the manufacture of polymer filaments, fibres, ribbons, sheets or moulded objects. It is theoretically possible to obtain materials with high tensile strength and toughness by engineering the orientation of the polymer molecules and the way in which they interact with one another. Strong, tough filaments, fibres or ribbons are useful in their own right for the manufacture, for example, of sutures, threads, cords, ropes, wound or woven materials. They can also be incorporated into a matrix with or without ether filler particles to produce tough and resilient composite materials. Sheets, whether formed from fibres or ribbons, can be stuck together to form tough laminated composites.

Natural silks are fine, lustrous filaments produced by the silkworm Bombyx mori and other invertebrate species. They offer advantages compared with the synthetic polymers currently used for the manufacture of materials. The tensile strength and toughness of the dragline silks of certain spiders can exceed that of Kevlar™, the toughest and strongest man-made fibre. Spider dragline silks also possess high thermal stability. Many silks are also biodegradable and do not persist in the environment. They are recyclable and are produced by a highly efficient low pressure and low temperature process using only water as a solvent.

The natural spinning process is remarkable in that an aqueous solution of protein is converted into a tough and highly insoluble material. The process in spiders and silkworms has been outlined in an article “Liquid crystalline spinning of spider silk” by Vollrath and Knight, Nature, vol 410, 29 Mar. 2002, pages 541-8. The authors not that lessons are to be learnt from the manner in which spiders and silkworms store their protein dope molecules and extrude them into strong threads. The article reviews the evidence that the natural spinning mechanism in spiders involves an addition of hydrogen ions and potassium ions and the removal of sodium ions as the spinning dope passes down the spinning duct.

It also reviews the evidence that natural spinning of proteins utilizes liquid crystalline feedstocks. The liquid crystalline state depends on a delicate balance between attractive and repulsive forces operating between molecules.

In an article by Knight and Vollrath “Biological Liquid Crystal Elastomers”, Trans. Phil. R. Soc. B. 357, 155-163 (2002), the authors note that much of the toughness of silks arise from the fact that they have a nanofibrillar composite construction; are constructed from very large amphiphilic repetitive block copolymers of the type ABAB, where A represents a hydrophobic block and B a less hydrophobic one; and are in essence liquid crystal elastomers.

The proteins spidroin and fibroin are found in two states: The first state is a safe storage state in which the extremely long protein chains are thought to be folded into fairly short and compactly folded rod-shaped molecules. The proteins in this first state are present in a highly concentrated aqueous solution and have a predominantly random coil and/or helical secondary structure. The second state is a solid state with a predominantly beta crystalline secondary structure. This second sate is a nanofibrillar composite, containing a high packing fraction of very long nanofibrils approximately 5 nm in diameter. The nanofibrils are oriented substantially parallel to the long axis of the tough thread and are thought to contain all or most of the beta crystallites. The beta crystallites have a width of about 5 nm and are arranged substantially parallel to the long axis of the nanofibrils. Small quantities of a less crystalline and more disordered material are thought to form the matrix between the nanofibrils.

The first storage state is found in the posterior and middle divisions of the gland in silkworms and in the analogous A-zone in spiders in which the protein is stored in a highly concentrated liquid crystalline state at remarkably high concentrations (20-40% w/v). In spiders, the protein is stored as ahigbly viscous liquid crystalline sol that persists through the first, second and most of third limb of the duct. In newly moulted final instar silkworms the protein is stored as a sol within the posterior and middle division of the gland, but is transformed into a gel in these divisions some while before the silkworm starts to spin. During this gel is converted back into a sol substantially close to where it enters the gland's duct. Thus in both cases, the spinning dope is present as a sol in order to flow through the duct of the gland. Knight and Vollrath have recently shown that in both silkworms and spiders, the thread is formed in a “drawn-down” process which initially commences within the lumen of the duct some distance before the point of discharge of the thread to the outside world.

In a PCT patent application No WO-A-01/38614, Knight and Vollrath have described an apparatus and method for forming materials that models the natural spinning of silk. This apparatus produces fibres and filaments by an extrusion method from dopes containing solutions of recombinant spider silk proteins or analogues or recombinant silk worn silk proteins or analogues or mixtures of such proteins or protein analogues or regenerated (redissolved) silk solution from silkworm silk. When these dopes are used in the apparatus it is necessary to store the dope prior to exclusion at a pH value above or below the isoelectric point of the protein to prevent the premature formation of insoluble material. According to this disclosure, other constituents may be added to the dope to keep the proteins or protein analogues in solution. These constituents may then be removed through the semipermeable and/or porous walls when the dope has reached the appropriate portion of the tubular passage in which it is desired to induce the transition from liquid dope to solid product, e. g. thread or fibre. The dope within the tubular passage can then be brought by dialysis against an appropriate acid or base or buffer solution to a pH value at or close to the pK value of one or more of the constituent proteins of the dope. Such a pH change will promote protein aggregation and the formation of an insoluble material. A volatile base or acid or buffer can also be diffused through the walls of the or each tubular passage from a vapour phase in the surrounding compartment or jacket to adjust the pH of the dope to the desired value. Vapour phase treatment to adjust the pH of the extruded material can also occur after the extruded material has left the outlet of the apparatus.

European patent application No. EP-A-0 072 024 (Whitney and Company) provides an example of the formation of a solid product from a liquid by gelation. In the Whitney patent application, a solid article of unsaturated polyester resin is formed in a mould by using a curing agent. The curing agent is a buffered equilibrium system of a relatively small quantity of un-ionised hydroperoxide and a salt soluble in the polyester resin, formed by the reaction of the hydroperoxide and a base. This curing agent also causes the polyester resin to gel. The teachings of the Whitney patent application are directed towards the production of moulded polyester articles and there is no suggestion that these teachings can be used in the production of moulded or extruded polypeptides or proteins.

UK Patent No. GB-A-1 539 725 (Unilever) shows an example of the production of a fibre containing a soy protein. The Unilever patent discloses the spinning of an aqueous solution of the soy protein to produce the soy fibre. Prior to or during the spinning process, the aqueous solution is heated to a temperature between 60° C. and 85° C. for a time sufficient to gel the con-glycinine fraction of the soy protein (the 7S fraction). After extrusion, the partially gelled material placed into a coagulating fluid medium in a bath which is hot enough to gel the glycinine fraction of the sol protein (11S fraction) This patent document states that the pH of the coagulating fluid medium is not a critical factor. In the two examples given in the patent document, the pH of the coagulating fluid medium is not varied.

An investigation of the gelling of some polypeptides has been published by Nowak et al “Rapidly recovering hydrogel scaffolds from self-assembling diblock copolypeptide amphiphiles”, Nature, Vol 417, 23 May 2002, pp 424-428. In this example, the gels are formed by changing the concentration of the polypeptides in water. There is no teaching of an extrusion method for the production of the solid material.

### SUMMARY OF THE INVENTION

It is therefore the object of the present invention to provide an improved apparatus and method means for extruding and forming objects made of proteins.

Accordingly, the invention is first directed to a method of forming an object from a feedstock made of a protein solution. The protein in the protein solution undergoes a sol-gel transition. The method c rises the following steps: a first step of adjusting the conditions of the feedstock to cause the feedstock to flow to form the object from the solution, with the protein substantially in the sol state, and a second step of adjusting the charge distribution on the protein and thus either to gel the feedstock or to bring it close to the sol-gel transition point. The step of adjusting the charge distribution can occur either at a different time or in a different location.

The inventors have discovered that protein solutions are best formed into objects at or around the sol-gel transition point of the protein.

The object may be formed in several ways generally know to the skilled person. The first step of forming the object may e.g. comprise a step of extruding, drawing, spinning or moulding the feedstock. In the case of moulding only after the object's desired shape has been attained will the charge distribution of the feedstock be adjusted so that the feedstock protein will transition to the gel state.

The protein used for forming the object can be a natural or synthetic protein, a glycoprotein, a phospho-protein, proteoglycan, or a mixture of two or more of these. The protein may be an amphiphilic block copolymer comprised of at least four perfect or imperfect repeats of at least two different types of blocks. At least one of these blocks should carry charged side chains to enable its assembly to be controlled by pH or by the addition of salts. It is thought that the provision of charged groups on a terminal domain or terminal domains of the protein may suffice.

The protein solution may comprise substantially fibroin, spidroin and/or mixtures or homologues thereof. The term “substantially”, as used in the present context, shall indicate that, while fibroin, spidroin and/or mixtures or homologues thereof or other amphiphilic block copolymer proteins or protein analogues be the principle component of the solution, further auxiliary or residual compounds may be present in the solution, as long as they do not substantially deteriorate the process of forming the object. The proteins may be natural proteins, genetically engineered proteins or natural or synthetic polypeptides.

In a preferred embodiment of the invention, it is useful to employ amphiphilic block copolymer molecules with some of the amino acid side chains capable of being charged such that each molecule can carry at least four negative or four positive unit charges or a combination of negative or positive charges.

In contrast to conventional gel forming polymers where a gel is formed by covalent bridges or by the addition of calcium or other metal ions to form electrovalent bridges between negatively charged groups, the gel of the invention is formed by changing the ionisation of charged groups on the protein by adjusting the pH or by shielding the charged groups on the polymer or by forming metal coordination complexes by the addition of salts or polyionic compounds to provide counter ions. The reduction in net charge on the polymeric molecules or aggregates thereof produced by changing or masking the ionisation of charged groups in this way will reduce the electrostatic repulsive forces operating between protein molecules or aggregates thereof. This allows the molecules to approach one another more closely and thus leading to a strengthening of the short range attractive forces. Alternatively, changing the ionisation of an amphoteric polymer molecule may permit the formation of salt bridges or metal coordination complexes resulting from the interaction of side groups with opposite electrostatic charge on adjacent chains. Thus, in either case, it is thought that weak interactions including hydrophobic and hydrophilic effects, Van der Waals and coulombic forces are responsible for an initial reversible gelation of the protein molecules. In the case of gelation of silk proteins or their analogues produced by a change in pH these weak interactions are strengthened with time by the spontaneous formation of numerous hydrogen bonds which form beta sheets. These effectively give rise to strong multivalent links within and between the protein molecules holding them together in a way that cannot easily be reversed.

Charged lyotropic liquid crystalline polymers are particularly useful for gel formation in tat the delicate balance between attractive and repulsive forces in this state of matter can be tipped in a reversible way towards greater attraction by manipulating electrostatic charges on the molecules.

The first step of adjusting the conditions of the feedstock may preferably comprise adjusting the pH, as it was found that the pH of the solution is one of the conditions responsible for the sol-gel transition. A buffer solution may be used to adjust the pH of the feedstock. The adjustment of the pH of the solution results in the alteration of charges on the molecules of the feedstock. In the case in which the polymer in the feedstock caries charged groups, the ions added to the feedstock will alter, neutralise or shield the charges on the polymer. As explained above, this permits the molecules to interact with one another as the result of non-covalent interactions. It was found to be of particular benefit if the buffer solution is selected from the group of buffer solutions comprising a small carboxylic acid such as formic acid, or acetic acid or propionic acid. This group of acids proved particularly effective for spinning. A small carboxylic acid has a molecular weight of less than 250 Daltons. Similarly, the addition of inorganic ions also can be effective in inducing the sol-gel transition or facilitating the formation of useful material.

It has been found that the addition of glycerophosphate to the buffer solution is particularly advantageous as it promotes the spinning of the polymer.

The present invention is also directed to an apparatus. The apparatus can, but is not limited to, carry out the above-summarised method.

The apparatus comprises a protein storage compartment for storing the protein in a sol condition and a transition compartment in which the sol-gel transition is induced.

The apparatus according to the invention may further comprise a compartment for holding the protein in contact with a buffer to keep it in a substantially sol condition. This feature will prevent premature sol-gel transition.

The inventive apparatus may further comprise a gel compartment for holding the protein in contact with a buffer to induce it to be substantially gelled. The buffer solution is selected from the group of buffer solutions described above that induce the sol-gel transition in the protein.

The inventive apparatus may further comprise a storage compartment for storing the protein solution in a sol condition subsequent before it is passed to the gel compartment.

### DESCRIPTION OF THE DRAWINGS

FIG. 1 is a schematic diagram of an apparatus for forming an object

FIG. 2 is a diagram showing an experimental setting for determining spinnability.

FIG. 3 is a diagram showing the effect of buffer type used.

### DETAILED DESCRIPTION OF THE INVENTION

Simultaneous observation of the spinnability and the state of the polymer feedstock (discussed below) lead to the conclusion at the protein is best spinnible at or substantially close to the transition point between its sol and gel states. This provides a means for determining the spinning conditions for many proteins or polypeptides, related polymers including charged amphiphilic block copolymers produced by chemical synthesis or by genetic engineering, or a combination of the two, and other charged polymers that undergo a sol-gel transition. Thus the sol-gel transition of many biopolymers can be switched reversibly by changing pH or other ions including small anions and cations.

It is believed to be particularly useful to employ amphiphilic block copolymer molecules, which carry at least four negative or four positive unit charges or a combination of negative and positive charges.

In contrast to covalently cross-linked gels such as formaldehyde cross-linked hyaluronan, gelatine or casein, or transaminase-cross-linked lactoglobulin, the gel of the invention is formed by changing the ionisation of charged groups on the protein by adjusting the pH or by shielding the charged groups on the polymer or by forming metal coordination complexes by the addition of salts or polyionic compounds to provide counter ions. The reduction in net charge on the polymeric molecules or aggregates thereof produced by changing or masking the ionisation of charged groups in this way will reduce the electrostatic repulsive forces operating between protein molecules or aggregates thereof. This allows the molecules to approach one another more closely and thus leading to a strengthening of the short range attractive forces. Alternatively, changing the ionisation of an amphoteric polymer molecule may permit the formation of salt bridges or metal coordination complexes resulting from the interaction of side groups with opposite electrostatic charge on adjacent chains. Thus, in either case, it is thought that weak interactions including hydrophobic and hydrophilic effects, Van der Waals and coulombic forces are responsible for an initial reversible gelation of the protein molecules. In the case of gelation of silk proteins or their analogues produced by a change in pH these weak interactions are strengthened with time by the spontaneous formation of numerous hydrogen bonds which form beta sheets. These effectively give rise to strong multivalent links within and between the protein molecules holding them together in a way that cannot easily be reversed.

It will be noted that charged lyotropic liquid crystalline polymers are particularly useful for gel formation in that the delicate balance between attractive and repulsive forces in this state of matter can be tipped in a reversible way towards greater attraction by manipulating electrostatic charges on the molecules.

It has been found that buffer solutions comprising a small carboxylic acid such as formic, acetic or propionic acid can be used to induce a sol-gel transition. In the case of acid addition the gelation is initially reversible but rapidly becomes irreversible with time. In contrast the addition of calcium or magnesium ions at concentrations between 50 and 500 mM in a neutral or alkaline buffer induces a gelation which remains reversible for a period of at least four weeks. Additionally, the addition of 0.0001-0,5M glycerophosphate improves the spinnability of the thread.

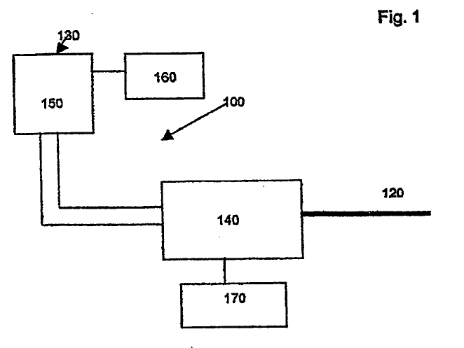

FIG. 1 shows a schematic diagram of a simple extrusion apparatus 100 for forming an object 120 according to the method of the invention. The extrusion apparatus 100 has a polymer storage compartment 130 in which the polymer 150 is stored in a sol condition. The extrusion apparatus 100 has furthermore a transition compartment 140 in which the polymer 150 undergoes a sol-gel transition and in which the object 120 is formed. The object 120 emerges from the transition compartment 140.

The sol-gel transition is induced in a number of ways. It had been noted (see above) that the spinnability of spider and silkworm protein was related to the pH of the protein The conditions in the transition compartment 140 were therefore adjusted by adjusting the pH of the polymer 150. This can be done in a number of ways such as by the addition of buffer salts or solutions, by dialysis against buffet solutions or by exposure to vapour from volatile buffers.

As noted above, it was found to be of particular benefit if the buffet solution is selected from the group of buffer solutions comprising a small carboxylic acid, such as forms acid, or acetic acid or propionic acid or. This group of acids proved particularly effective in inducing the sol-gel transition. It is thought that the hydrogen ions react with the negatively charged carboxyl groups on the side chains of aspartic and glutamic acid to reduce the negative charge of the protein permitting initially reversible aggregation resulting from the formation of a small number of weak intermolecular interactions. This is followed by the more gradual secondary structural transition to the beta-sheet form resulting in irreversible gel formation.

It was also found to be of particular benefit to add potassium or sodium ions to the protein, preferably as a chloride.

It was also found to be of additional benefit to add small quantities of a small molecular weight polyol to the polymer solution. In the preferred embodiment glycerophosphate disodium salt is added to a final concentration of 0.0001 to 0.1 M.

In another example, it has been found that the addition of calcium ions to the protein induces the sol-gel transition. It appears that the addition of the calcium ions results in the formation of co-ordination complexes between calcium ions and the oxygen atoms of carboxyl groups' side chains of aspartic acid. These complexes can form intermolecular links between two to four peptide chains.

The polymer storage compartment 130 is connected to a first buffer compartment 160 that contains a first buffer solution. The first buffer solution could be, for example, ammonia. The first buffer solution is designed to keep the polymer 150 in a sol condition.

The transition compartment 140 is connected to a second buffer compartment 170 that contains an acidic solution to cause gelling of the polymer 150. Suitable acidic solutions include, hut are not limited to, small carboxylic acids such as formic acid, acetic acid, or propionic acid

In an embodiment of the invention the extrusion apparatus 100 includes a further storage compartment that stores the polymer 150 in a sol condition.

It is to be noted that the use of an extrusion die as the transition compartment 140 with one or more surfaces of the inner passage of the die made semi-permeable or porous can be used to effect a sol gel transition in appropriate polymers to assist in the spinning or extrusion of the polymer. By way of example only, such an extrusion die is described in patent application WO 01/38614 (Vollrath and Knight), the teachings of which are incorporated herein by reference. It is also to be noted that the control of the sol-gel transition enables polymers to flow or be blown into the transition compartment 140 in a mould in the sol state and converted into the gel state by providing the mould with one or more semi-permeable or porous surfaces through which a gelling agent (such as an acid or calorie ions) can be introduced or by opening one or more surfaces of the mould to expose the material to the gelling agent. Alternatively, a mould with sides and bottom but no top can be used and the gelling agent applied though the open upper side.

After gelling, the protein is cross-linked using a cross-linking agent such as glutaldehyde or carbodiimide. It is advantageous to introduce additional steps after gelling and before cross-linking and after cross-linking to remove more water from the gelled protein. This can be achieved by reverse dialysis or simply by air drying.

### EXAMPLE 1

### Experimental Method for Determining the Conditions for Spinning Fibres

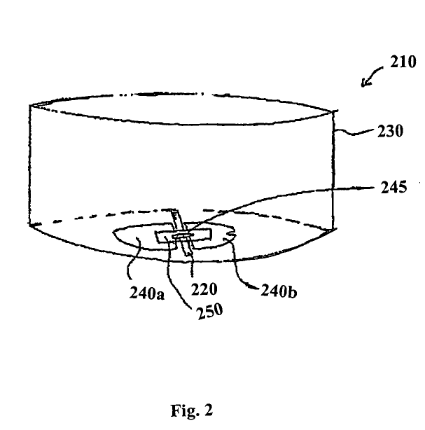

The conditions required for optimum spinning of protein dopes were determined using the apparatus 210 shown in FIG. 2. A polycarbonate spacer 220 (about 1 mm wide and 0.5 mm thick) is first stuck to the midline in the bottom of a 53 mm plastic Petri dish 230 to form a waterproof barrier. A filter paper 240 (Whatman I and having a diameter of 42.5 mm) is cut into two halves 240a, 240b. One filter paper half 240a is moistened with a suitable buffer solution, for example, 0.1 M Trizma/HCl buffer at pH 7.0 for keeping the protein dope in the sol state The moistened filter paper half 240a is then placed on the left band side of the polycarbonate spacer 220 as shown in FIG. 1. The other filter paper half 240b is notched to indicate that this is the low pH side and is moistened with the test solution (a buffer solution of pH<5.5 with or without other dopants) before being placed on the right hand side of the polycarbonate spacer 220.

A 10 mm square of dialysis membrane 250 (made by Visking and having an exclusion limit 18-20 kDa) is placed on top of the two moistened filter paper halves 240a, 240b with its centre over the centre of the Petri dish 230. 5 to 20 μl of highly concentrated test protein (approximately 20% w/v) are placed in an elongated drop 245 on the top surface of the dialysis membrane 250 in such a war that a pH gradient is established across the elongated drop 245. The protein selected was either spidroin or fibroin obtained by the dissection of glands form spiders or the larvae of Lepidopteran insects respectively. It is to be noted that gradients of pH and other diffusible ions and diffusible small molecules can be set up by diffusion through the dialysis membrane 250 into the elongated drop 245 of protein. This makes it possible to mimic the gradients between the protein dope storage and spinning regions of the silk duct in spiders, silkworms and other arthropods.

The apparatus 210 is left undisturbed for a defined period of between 5 and 30 minutes to give time for the establishment of the pH gradient Thereafter the end of the elongated drop 245 of the protein over the fight band filter paper 240b is seized with watchmaker's forceps (DuMont Number 55 made of stainless steel) and slowly pulled from left to right in an attempt to draw out a fibre 260. The maximum length of thread in centimeters, which can be drawn, is a measure of the spinnability of the fibre 260. The condition of the protein dope on the fight hand side of the elongated drop 245 is also assessed by eye using a stereo microscope. If only a short cone of protein dope can be drawn out that flows back into the elongated drop 245 after rupture, the protein dope in the elongate drop 245 is preset as a sol. We further assessed the viscosity of the sol by determining how long it takes the cone to flow back into the elongated drop. If, on the other and, the protein dope is found to have a stiff rubbery consistency and little or no material can be pulled out from it, recoiling sharply when released, the protein dope is described as a solid gel. The temperature is maintained at 20° C. throughout the example.

The apparatus 210 is used to test the hypothesis that the spinnability of spider and silkworm protein removed directly from the A-zone in spiders and from he anterior and median division in silkworms was dependent on pH and potassium ion concentration. The results showed both dopes showed a marked improvement in spinnability at optimum pH values and potassium ion concentrations. It was discovered that the exposure of the protein dope to buffers of different pH revealed a remarkably sharp sol-gel transition as the pH was lowered. This transition generally occurred at a pH value between 4.0 and 6.5 that depended on the nature of the buffer used and the concentration of potassium and other ions. The addition of potassium chloride at concentrations of 50 to 500 mM produced a marked increase in the pH for the sol-gel transition point and improved spinnability. The validity of this simple method was confirmed for fibroin by spinning in a more complex biomimetic spinning device of the type described in PCT application WO-A-01/38614

In the course of these investigations it was also discovered that optimum spinnability in the simple device occurred substantially close to the sol-gel transition point It was also discovered that exposure of both protein dopes of spider and silkworm protein to acetic acid vapour from 0.01 M and glacial acetic acid at 20 to 30 degrees Centigrade produced a rapid conversion of concentrated dope sols to stiff rubbery gels in both organisms. The change could be rapidly reversed by exposure of the protein dope to ammonia vapour provided that the protein bad only been exposed to the acid for short periods of time (less than 10 minutes). Under these conditions the change appeared to be substantially reversible and could be obtained at least tour times by repeated alternate application of acetic acid and ammonia vapours. The use of the simple apparatus of FIG. 2 also enabled the discovery of the optimum conditions for spinnability and the effect of adding dopants to improve spinnability of silkworm and spider dope solutions.

These observations suggest that charged groups on the protein polymers are responsible for the pH dependent sol-gel transition in spidroins and fibroins and tat other charged repetitive amphiphilic block copolymers could be used in place of these proteins.

Five factors were shown to influence spinnability: buffer type, pH concentration of inorganic ions, and duration of exposure to buffets in the apparatus the addition of glycerophosphate and The addition of other dopants. However, further factors influencing the spinnability may be discovered and these should be understood to be comprised within the scope of the present invention.

### EXAMPLE 2

### Effect of Buffer Type on Spinnability

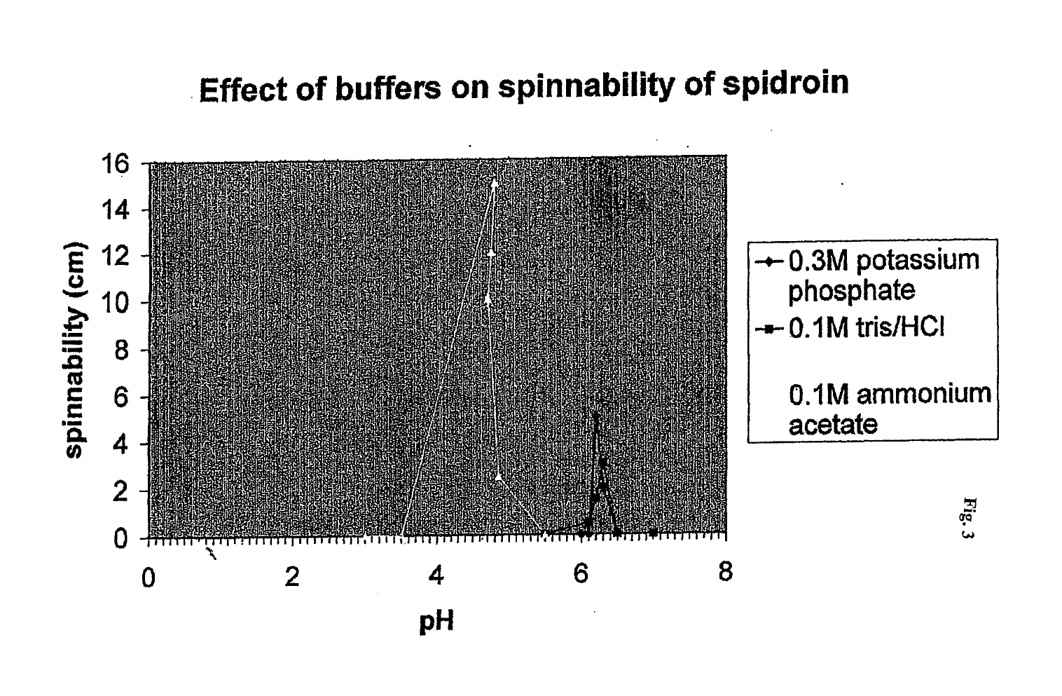

FIG. 3 shows typical results showing the effect of the type of buffer solution used to moisten the right hand filter paper 240b of FIG. 2 on spinnability.

For fibroin and spidroin proteins, the spinnability decreased in the following order:

Ammonium acetate>ammonium formate>ammonium propionate>>>potassium phosphate>tris/HCl (tris (hydroxymethyl)aminomethane/HCL)=HEPES (4-(2-hydroxyethyl)-I-piperazine ethane-sulfonic acid)=PIPES (1,4-piperazinbis(ethanesulfonic acid>>. Thus monocarboxylic acids with a short chain length appeal to be the best buffer solutions. In this connection it is of interest to note that other proteins, such as collagens and high molecular weight glutenins, show a higher solubility in acetate buffer solutions compared with other buffer systems. These proteins resemble silk proteins in that they are repetitive amphiphilic block copolymers with a pI above 7.0. This suggests a preferential interaction of acetate ions with the proteins under acidic conditions, possibly dependent on the small size of this carboxylic ion.

### EXAMPLE 3

### Effect of pH an Spinnability

The pH value for obtaining the optimum spinning conditions for some of the buffer solutions was investigated.

The optimum pH value for spinning spidroin from ammonium acetate buffer solution was pH 4.7±0.1, that of ammonium formate approximately pH 4.8 whilst the optima for the other buffer solutions was about pH 6.3. The latter values are close to the estimated pH in the duct of the spider and the pH of phosphate buffer solution for maximum sensitivity to shear in dilute spidroin solutions, it is also close to the pK for the natural buffering of spidroin dope freshly removed from the spider's silk gland. Histidine groups in proteins protonate between pH 6 and 7 strongly suggesting that the observed pK, optimal sensitivity to shear and sol-gel transition (see below) for spidroin in phosphate buffer involve the protonation of histidine groups. The pH optimum for spinning silkworm fibroin in an ammonium acetate buffer solution is 4.8±0.2 and in an ammonium formate buffer solution is (5.2±0.1).

### EXAMPLE 4

### Duration of the Action of the Buffer Solution

The time dependency of structural changes in the test proteins together with the time taken for the diffusion of ions cross the dialysis membrane 250 means that spinnability and optimum pH value are affected by time of exposure of the protein dope to the conditions above the fight hand filter paper 40a. Using indicators, it was shown that pH equilibration is substantially complete after 5 minutes at 20° C. This means that the results obtained with the simple apparatus shown in FIG. 2 are only a guide to the pH conditions required in spinning in a more sophisticated extrusion or spinning device involving the diffusion of buffer solutions or acid vapours into extrusion or spinning solutions. Thus pH optima will have to be varied somewhat according to conditions affecting rate of diffusion including temperature, nature of diffusion barriers, geometry of die and rate of extrusion or spinning, as will be understood by a person skilled in protein biophysics.

### EXAMPLE 5

### Formation of Moulded Articles Firm Proteins

One or more concentrated (10-75% w/v) solutions of fibroin or spidroin were prepared as follows:

In a first step, native fibroin was taken directly from Bombyx mori silk glands (as disclosed in international patent application No. WO-A-03/037925 (Vollrath). The native fibroin is concentrated in a dialysis bag (MWCO 5-8 KDa) by reverse dialysis for 12 hrs or overnight 8 at 4° C. against a solution condoning (in final concentrations) 20-40% w/v polyethylene glycol (MW 15-20 kDa), 0.1 M ammonium acetate buffer at pH 7.

The resulting silk solutions should have a concentration of 10-75% which can be varied by altering the concentration of the polyethylene glycol and or the length of reverse dialysis. The pH of the final solution should be between pH 7 and 8.5.

The concentrate protein solution was then placed into a mould in which the top is open at or in which at least one surface of the mould was formed by a porous or semipermeable material to enable the concentrated protein to be converted to a gel. The concentrated protein in the mould was then gelled by exposure to either acetic acid vapour (5 minutes to 3 hours at ambient temperature) or 0.1 M to 0.5M acetic acid solution (10 minutes to 1 hour).

Alternatively the protein can be gelled by exposure to a solution containing (final concentrations) 0.1 M ammonium acetate buffer pH at 7.8 and 50 to 750 calcium ions preferably as the chloride for 5 minutes to 3 hours at ambient temperature.

The gelled concentrated protein can be cross-linked in the mould by a carbodiimide solution or an aldehyde solution or vapour as will be understood by a person skilled in the art

### EXAMPLE 6

In this example, native fibroin was taken directly from Wild Silk moth glands and concentrated and moulded as described in Example 5.

### EXAMPLE 7

In this example, saturated aqueous solutions of regenerated fibroin solutions was prepared by dissolving degummed Bombyx mori or Wild Silkworm (Tussah silk) in said aqueous lithium bromide solutions and dialysing this solution for 12 hours at 4° C. against distilled water (at least three changes) followed by concentration by reverse dialysis for 12 hours at 4° C. against excess of a solution containing polyethylene glycol and ammonium acetate as described in Example 5 but containing in addition a final concentration of 0.5 M lithium bromide.

Moulded samples were prepared as in Example 5.

1. A method of forming an object (120) from a feedstock (150;245) made of a protein solution of a protein in sol state, the protein undergoing a sol-gel transition, and comprising the following steps:
a first step of adjusting the conditions to cause the feedstock to flow to form the object (120); and
a second step of adjusting the conditions of the feedstock (150;245) to adjust the charge distribution on the protein and thereby either gelling the feedstock (150) or to bringing the feedstock (150) close to the sol-gel transition point.

2. The method of claim 1, wherein the first step of forming the object (120) comprises a step of extruding, spinning or moulding the feedstock (150;245).

3. The method of claim 1, wherein the protein is a natural or synthetic protein, proteoglycan, glycoprotein or phospho-protein or a mixture of these.

4. The method of claim 1 or 2, wherein the protein is fibroin, spidroin or a homologue thereof.

5. The method of claim 1 or 2, wherein the protein is an amphiphilic block copolymer comprised of at least four perfect or imperfect repeats of at least two different types of blocks.

6. The method of any one of the above claims claim 1, wherein the protein carries at least four unit negative or positive charges.

7. The method of any one of the above claims claim 1, wherein the second step of adjusting the conditions on the feedstock (150;245) is carried out by adding a counter ion to the feedstock (150;245).

8. The method of any one of the above claims claim 1, wherein the second step of adjusting the conditions of the feedstock (150;245) comprises adjusting the pH of the feedstock (150;245).

9. The method of claim 8 comprising the use of a buffer solution to adjust the pH of the feedstock (150;245).

10. The method of claim 9 wherein the buffer solution is selected from the group of buffer solutions comprising a small carboxylic acid such as formic acid, or acetic acid or propionic acid.

11. The method of claim 9 or 10, wherein inorganic ions are added to the buffer solution.

12. The method of claims 9 to 11 claim 9, wherein glycerophosphate is additionally added to the buffer solution.

13. The method of claims 1 to 12 claim 1, wherein the second step of adjusting the conditions of the feedstock (150;245) comprises a step of adding metal ions to the feedstock (150;245).

14. The method of claim 13, wherein the metal ions are selected from the group consisting of magnesium ions and calcium ions.

15. The method of claim 13, wherein the metal ion is a calcium ion.

16. The method of any one of the above claims claim 1, further comprising a third step of cross-linking the protein in the gel state.

17. The method of any one of the above claims) claim 1, further comprising a dehydration step of dehydrating the protein in the gel state.

18. Use of a buffer solution selected from the group of buffer solutions comprising a small carboxylic acid, acetic acid, cid or propionic acid to induce a sol-gel transition in a polypeptide solution (150;245).

19. Use of a metal ion selected from the group of metal ions consisting of magnesium and calcium to induce a sol-gel transition in a polypeptide solution (150;245) The use of claim 18, wherein the polypeptide solution comprises substantially fibroin, spidroin and/or mixtures homologues thereof.

20. The use of claim 18 or 19 wherein the polypeptide solution comprises substantially fibroin, spidroin and/or mixtures or homologues thereof. Use of a metal ion selected from the group of metal ions consisting of magnesium and calcium to induce a sol-gel transition in a polypeptide solution.

21. An apparatus (100;210) for forming an object (120;260) from a feedstock made of a protein, the protein undergoing a sol-gel transition at a transition point at transition conditions, the apparatus comprising:
a protein storage compartment (130) for storing the protein in a sol condition; and
a transition compartment (140) in which the sol-gel transition is induced by changing the charge distribution on the protein using a gelling agent. The use of claim 20 wherein the polypeptide solution comprises substantially fibroin, spidroin and/or mixtures or homologues thereof.

22. The apparatus of claim 21 wherein the protein storage compartment (130) holds the protein in contact with a buffer solution to keep the protein in a substantially sol condition. An apparatus for forming an object from a feedstock made of a protein, the protein undergoing a sol-gel transition at a transition point at transition conditions, the apparatus comprising:
a protein storage compartment for storing the protein in a sol condition; and
a transition compartment in which the sol-gel transition is induced by changing the charge distribution on the Protein using a gelling agent.

23. The apparatus of claims 21 to 22, wherein the protein solution comprises a natural or synthetic protein, proteoglycan, glycoprotein or phospho-protein or a mixture of at least two of these. The apparatus of claim 22 wherein the protein storage compartment holds the protein in contact with a buffer solution to keep the protein in a substantially sol condition.

24. The apparatus of claims 21 to 22, wherein the protein is an amphiphilic block copolymer comprised of at least four perfect or imperfect repeats of at least two different types of blocks. The apparatus of claim 22, wherein the protein solution comprises a natural or synthetic protein, proteoglycan, glycoprotein or phospho-protein or a mixture of at least two of these.

25. The apparatus of claims 21 to 22, wherein the protein carries at least four unit negative or positive charges. The apparatus of claim 22, wherein the protein is an amphiphilic block copolymer comprised of at least four perfect or imperfect repeats of at least two different types of blocks.

26. The apparatus of claim 21 to 25, wherein the buffer solution is selected from the group comprising a small carboxylic acid, such as acetic acid, or propionic acid. The apparatus of claim 22, wherein the protein carries at least four unit negative or positive charges.

27. The apparatus of one of claims 21 to 26, wherein the protein solution is a solution of fibroin, spidroin or a homologue thereof. The apparatus of claim 23, wherein the buffer solution is selected from the group comprising a small carboxylic acid, such as acetic acid, or propionic acid.

28. The apparatus of one of claims 21 to 27, wherein the apparatus (100;210) further comprises a compartment for holding the protein in contact with a second buffer solution to induce it to be substantially gelled, the second buffer solution being selected from the group comprising solutions that induce the sol-gel transition in the protein. The apparatus of claim 22, wherein the protein solution is a solution of fibroin, spidroin or a homologue thereof.

29. The apparatus of one of claims 21 to 28 further comprising a further storage compartment (180) for storing the protein solution in a sol condition subsequent to it being past to the transition compartment (140). The apparatus of claim 22, wherein the apparatus further comprises a compartment for holding the protein in contact with a second buffer solution to induce it to be substantially gelled, the second buffer solution being selected from the group comprising solutions that induce the sol-gel transition in the protein.

30. The apparatus of one of claims 21 to 29, wherein the gelling agent comprises counter-ions. The apparatus of claim 22 further comprising a further storage compartment for storing the protein solution in a sol condition subsequent to it being past to the transition compartment.

31. The apparatus of claim 30, wherein the counter ions are either hydrogen ions or metal ions. The apparatus of claim 22, wherein the gelling agent comprises counter-ions.

32. The apparatus of claim 31, wherein the metal ions are selected from the group consisting of magnesium ions and calcium ions. The apparatus of claim 31, wherein the counter ions are either hydrogen ions or metal ions.

33. The apparatus of claim 32, wherein the metal ions are selected from the group consisting of magnesium ions and calcium ions.
